(19) United States
(12) Patent Application Publication
(10) Pub. No.: US 2024/0062374 A1
(43) Pub. Date: Feb. 22, 2024

(54) MEDICAL INFORMATION PROCESSING DEVICE, MEDICAL INFORMATION PROCESSING METHOD, AND RECORDING MEDIUM

(71) Applicants: CANON MEDICAL SYSTEMS CORPORATION, Otawara-shi (JP); Canon Kabushiki Kaisha, Tokyo (JP)

(72) Inventors: Kohei WATANABE, Utsunomiya (JP); Katsuhiko FUJIMOTO, Saitama (JP); Atsuko SUGIYAMA, Nasushiobara (JP)

(73) Assignees: CANON MEDICAL SYSTEMS CORPORATION, Otawara-shi (JP); Canon Kabushiki Kaisha, Tokyo (JP)

(21) Appl. No.: 18/452,114

(22) Filed: Aug. 18, 2023

(30) Foreign Application Priority Data
Aug. 22, 2022 (JP) 2022-131892

(51) Int. Cl. G06T 7/00; G16H 30/40

(52) U.S. Cl. CPC G06T 7/0012; G16H 30/40; G06T 2207/30068; G06T 2207/30024

(57) ABSTRACT

The medical information processing device according to the embodiments includes processing circuitry configured to determine a resection target portion in a medical image depicting a target site of resection by surgical treatment, and calculate, based on the determined resection target portion, an amount of cells to be produced.

### CROSS-REFERENCE TO RELATED APPLICATIONS

This application is based upon and claims the benefit of priority from Japanese Patent Application No. 2022-131892, filed on Aug. 22, 2022, the entire contents of which are incorporated herein by reference.

### FIELD

Embodiments described herein relate generally to a medical information processing device, a medical information processing method, and a recording medium.

### BACKGROUND

In recent years, application of treatment using stem cells such as adipose-derived stem cells and iPS cells has expanded. For example, hereditary breast and ovarian cancer (HBOC) syndrome is now covered by insurance from a test to prophylactic resection and reconstruction, and regenerative medicine is being used for breast reconstruction.

In this context, breast reconstruction using stem cells requires a culture period to prepare the cell mass required for reconstruction for patients with less fat because, for example, fewer cells can be harvested therefrom. Therefore, it is required to supply an appropriate amount of cells according to a reconstruction timing.

### BRIEF DESCRIPTION OF THE DRAWINGS

FIG. 1 is a diagram illustrating an example of a configuration of a treatment support system according to an embodiment;

FIG. 2 is a diagram illustrating an example of a configuration of a support device according to the embodiment;

FIG. 3 is a flowchart illustrating an example of workflow of treatment support according to the embodiment;

FIG. 4 is a flowchart illustrating an example of the support processing flow executed in the support device according to the embodiment;

FIG. 5 is a flowchart illustrating an example of workflow for cell culture in the treatment support according to the embodiment;

FIG. 6 is a flowchart illustrating another example of the support processing flow executed in the support device according to the embodiment;

FIG. 7 is a flowchart illustrating another example of the workflow for cell culture in the treatment support according to the embodiment;

FIG. 8 is a flowchart illustrating another example of the workflow for cell culture in the treatment support according to the embodiment;

FIG. 9 is a flowchart illustrating another example of the flow of the support processing executed in the support device according to the embodiment; and

FIG. 10 is a flowchart illustrating another example of the workflow for cell culture in the treatment support according to the embodiment.

### DETAILED DESCRIPTION

A medical information processing device described in the following embodiments includes processing circuitry configured to determine a resection target portion in a medical image depicting a target site of resection by surgical treatment and to calculate, based on the determined resection target portion, an amount of cells to be produced.

A medical information processing device, a medical information processing method, a computer program, and a recording medium according to each of the embodiments will be described with reference to the drawings below. In the following description, components that have the same or substantially the same functions as those precedingly described with respect to the figures already illustrated are indicated with the same symbols and explained in duplicate only when necessary. Even when the same part is represented, dimensions and proportions may differ from each other depending on the drawing. For example, from a viewpoint of ensuring the visibility of the drawings, reference codes are attached only to major components in the description of each drawing, and the reference codes are not attached to components having the same or substantially the same functions in some cases.

In recent years, application of treatment using stem cells such as adipose-derived stem cells and iPS cells has expanded. For example, hereditary breast and ovarian cancer (HBOC) syndrome is now covered by insurance from a test to prophylactic resection and reconstruction, and regenerative medicine is being used for breast reconstruction.

In this context, breast reconstruction using stem cells requires a culture period to prepare the cell mass required for reconstruction for patients with less fat because, for example, fewer cells can be harvested therefrom. Therefore, it is required to supply an appropriate amount of cells according to a reconstruction timing.

In addition, after reconstruction (primary reconstruction), additional reconstruction (secondary reconstruction) was required because transplanted cells were not engrafted and were necrosed, or the transplanted cells were absorbed to change a morphology of a breast over time.

Therefore, the present disclosure discloses a treatment support system 1 that can appropriately support reconstruction of a target site by cell transplantation.

### First Embodiment

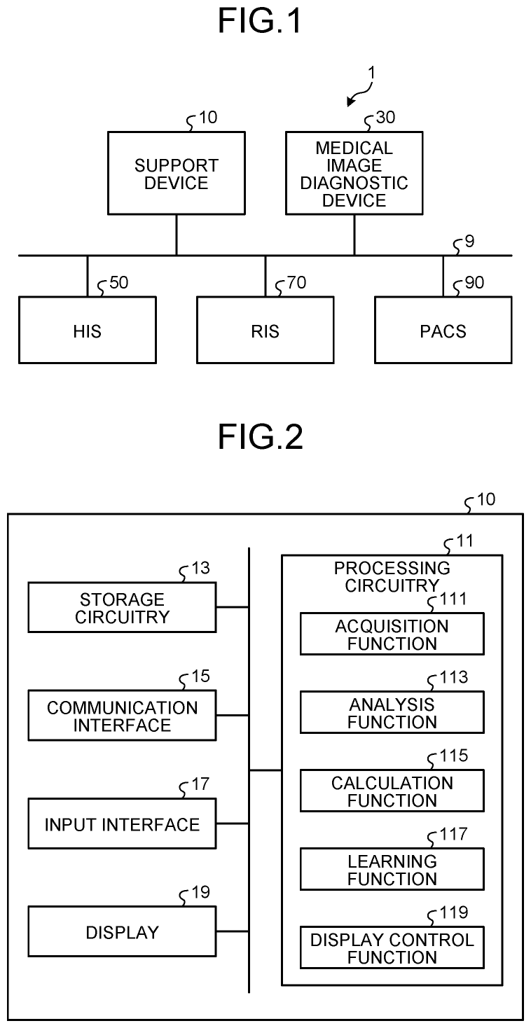

FIG. 1 is a diagram illustrating an example of a configuration of the treatment support system 1 according to an embodiment. As illustrated in FIG. 1, the treatment support system 1 includes a support device 10, a medical image diagnostic device 30, a hospital information system (HIS) 50, a radiology information system (RIS) 70, and a medical image management system (PACS: Picture Archiving and Communication Systems) 90. Each of the devices in the treatment support system 1 is installed in a hospital, for example, and can communicate with other devices through a network 9 such as an in-hospital local area network (LAN). The HIS 50 may be connected to an external network in addition to the in-hospital LAN. Here, the support device 10 is an example of the medical information processing device.

The HIS 50 is a system that manages information generated in the hospital. Information generated in the hospital includes patient information and test order information. Records included in the patient information each have the following items: a patient ID, a patient name (name), age (date of birth), sex, height, weight, a blood type, and the like. Records included in the test order information each have the following items: a test ID that identifies the test, a patient ID, information indicating an inpatient or outpatient status, a test code, a medical specialty, a test type, a test site, a scheduled test date, and the like.

The test ID is an identifier that is issued when test order information is entered, and that uniquely identifies test order information, for example, within one hospital. The patient ID is an identifier that is correspondingly assigned to a patient, and that uniquely identifies the patient within one hospital, for example. The test code is an identifier that is defined, for example, within one hospital, and uniquely identifies a test. The medical specialty, for example, indicates a specialty category of medical treatment in medicine. Specifically, the medical specialty indicates internal medicine, surgery, or the like. The test type indicates a test using medical images. For example, the test type includes an X-ray test, a computed tomography (CT) test, a magnetic resonance imaging (MRI) test, or the like. The test site includes a brain, a kidney, a lung, a liver, or the like.

When test order information is entered by a test ordering physician, for example, the HIS 50 transmits the entered test order information and patient information identified by the test order information to the RIS. In this case, the HIS 50 also transmits the patient information to the PACS.

The RIS 70 is a system that manages test reservation information related to radiograph test services. For example, the RIS 70 receives test order information transmitted from the HIS 50, adds various types of setting information to the received test order information to accumulate, and manages the accumulated information as test reservation information. Specifically, when receiving patient information and test order information transmitted from the HIS 50, the RIS 70 generates test reservation information necessary to operate the medical image diagnostic device 30, based on the received patient information and the received test order information. The test reservation information includes information necessary to perform the test, for example, a test ID, a patient ID, a test type, and a test site. The RIS 70 sends the generated test reservation information to the medical image diagnostic device 30.

The medical image diagnostic device 30 is a device that generates medical image data based on data collected from a subject. As the medical image diagnostic device 30, various medical image diagnostic devices can be used, such as an X-ray diagnostic device, an X-ray computed tomography (CT) device, a magnetic resonance imaging (MRI) device, an ultrasonic diagnostic device, a single photon emission computed tomography (SPECT) device, a positron emission computed tomography (PET) device, a SPECT-CT device having the SPECT device and the X-ray CT device integrated therein, a PET-CT device having the PET device and the X-ray CT device integrated therein, or the like.

The medical image diagnostic device 30 performs the test based on the test reservation information transmitted from the RIS 70, for example. The medical image diagnostic device 30 generates and transmits to the RIS 70 test performance information indicating implementation of the test. In this case, the RIS 70 receives the test performance information from the medical image diagnostic device 30 and outputs the received test performance information to the HIS 50 or other devices as the latest test performance information. For example, the HIS 50 receives the latest test performance information and manages the received test performance information. The test performance information includes test reservation information such as the test ID, patient ID, the test type, and the test site, and performance date and time of the test.

The medical image diagnostic device 30 converts generated medical image data into a format conforming to the Digital Imaging and Communication in Medicine (DICOM) standard, for example. Specifically, the medical image diagnostic device 30 generates medical image data having a DICOM tag added thereto as supplementary information.

The supplementary information includes, for example, the patient ID, the test ID, a device ID, an image series ID, and the like, and is standardized according to the DICOM standard. The device ID is information for identifying the medical image diagnostic device 30. The image series ID is information for identifying a single imaging by the medical image diagnostic device 30 and includes, for example, an imaged site of the subject (patient), the time of image generation, a slice thickness, a slice position, and the like. For example, by performing a CT test or an MRI test, it is possible to obtain corresponding tomographic images at a plurality of the slice positions as medical image data.

The medical image diagnostic device 30 transmits the generated medical image data to the PACS 90. The PACS 90 is a system that manages various types of medical image data.

The PACS 90, for example, receives patient information transmitted from the HIS 50 and manages the received patient information. The PACS 90 includes storage circuitry configured to manage patient information. The PACS 90, for example, receives medical image data transmitted from the medical image diagnostic device 30, and associates the received medical image data with the patient information to store in the storage circuitry of the PACS 90. Note that pieces of the supplementary information such as the patient ID, the test ID, the device ID, and the image series ID are added to the medical image data stored in the PACS 90. Therefore, the operator can acquire necessary patient information from the PACS 90 by performing a search using the patient ID or the like. Moreover, the operator can acquire necessary medical image data from the PACS 90 by performing a search using the patient ID, the test ID, the device ID, the image series ID, or the like.

Here, the HIS 50 receives, for example, an electronic medical record prepared by a clinician who is a physician requesting the test and test performance information corresponding to the electronic medical record, and associates the received electronic medical record with the received test performance information to store in the storage circuitry of the HIS 50. As described above, the test performance information includes the test ID, the patient ID, the test type, the test site, performance date and time of the test, and the like, and thus the operator can acquire necessary electronic medical record from the HIS 50 by performing a search using the patient ID, the test ID, or the like. Here, in the present embodiment, the electronic medical records are stored in the storage circuitry of the HIS 50, but may be stored in storage circuitry of other devices in the treatment support system 1, as long as a search by ID is enabled.

Furthermore, the RIS 70 receives, for example, a radiogram interpretation report created in response to input by a radiologist and test performance information corresponding to the radiogram interpretation report, and associates the received radiogram interpretation report with the received test performance information to store in the storage circuitry of the RIS 70. As described above, the test performance information includes the test ID, the patient ID, the test type, the test site, performance date and time of the test, and the like, and thus the operator can acquire necessary radiogram interpretation report from the RIS 70 by performing a search using the patient ID, test ID, or the like. Here, in the present embodiment, the radiogram interpretation report is stored in the storage circuitry of the RIS 70, but it may be stored in storage circuitry of other devices in the treatment support system 1 as long as a search by ID is enabled.

The support device 10 executes the support processing. The support device 10 acquires various medical data from the medical image diagnostic device 30, the HIS 50, the RIS 70, and the PACS 90 via the network 9, and performs various types of information processing using the acquired medical data. For example, the support device 10 is implemented by a computer such as a workstation having a processor and a memory, such as ROM, RAM, or the like, as hardware resources. The support device 10 implements, for example, an integrated viewer. The integrated viewer is an application that presents medical information to a user in an integrated manner. The integrated viewer may adopt any desired implementation form, such as a web application, a fat client application, a thin client application, or the like.

The medical data is information indicating medical records that medical professionals can obtain regarding physical conditions, medical conditions, treatment, and the like of the patient in a course of medical care. The medical data includes data acquired in various environments with, for example, devices from different manufacturers, different versions of devices, different settings for the same device, or the like. The medical data is not limited to objective data such as numerical values, but may also include non-numerical, for example, subjective data expressed in text. The medical data includes, for example, test history information, image information, electrocardiogram information, vital sign information, medication history information, report information, medical record information, nursing record information, referral letters, and discharge summaries. The test history information is information that represents a history of test results acquired as a result of specimen and bacteriological tests performed on the patient, for example. The image information is information indicating locations of medical images acquired by, for example, imaging the patient. The image information includes, for example, information indicating the location of a medical image file generated by the medical image diagnostic device as a result of the test having been performed. The electrocardiogram information is, for example, information about an electrocardiogram waveform measured from the patient. The vital sign information, for example, is basic information about the life of the patient. The vital sign information includes, for example, pulse rate, respiratory rate, body temperature, blood pressure, and level of consciousness. The medication history information is, for example, information indicating a history of amounts of medication administered to the patient. The report information is information obtained by summarizing conditions and a disease of the patient after a radiologist of a department of radiology reads medical images such as X-ray images, CT images, MRI images, and ultrasound images in response to a test request from a physician in a hospital department, for example. The report information includes, for example, radiogram interpretation report information that represents a radiogram interpretation report created by the radiologist referring to a medical image file stored in the PACS. The report information includes information indicating the patient ID, patient name, and date of birth of the patient corresponding to a medical image file for radiogram interpretation, for example. The medical record information is information that a physician has inputted to an electronic medical record, for example. The medical record information includes, for example, a medical record during hospitalization, and medical and medication histories of the patient. The nursing record information is information that a nurse or the like has inputted to the electronic medical record, for example. The nursing record information includes nursing records during hospitalization. The nursing record information may include a feeding record during hospitalization. The medical data may further contain information about accounting.

The treatment support system 1 may have Vendor Neutral Archive (VNA) systems instead of the HIS 50, the RIS 70 and the PACS 90. The VNA system is an integrated archiving system that centrally manages a variety of medical data managed by the PACS 90 from a different manufacturer and each of the systems (HIS 50, RIS 70) of different clinical departments. The VNA system is communicatively connected to, for example, the HIS 50, the RIS 70, and the PACS 90, with each other via a LAN or other in-hospital network. The various types of information managed and stored by the VNA system is not necessarily acquired from systems made by different manufacturers, but may be acquired from systems made by a single manufacturer.

FIG. 2 is a diagram illustrating an example of a configuration of the support device 10 according to the embodiment. The support device 10 includes processing circuitry 11, a storage circuitry 13, a communication interface 15, an input interface 17, and a display 19, as illustrated in FIG. 2. The processing circuitry 11, the storage circuitry 13, the communication interface 15, the input interface 17, and display 19 are communicatively connected via a bus or other means.

The storage circuitry 13 stores various data. For example, the storage circuitry 13 stores image data received from the medical image diagnostic device 30, the HIS 50, the RIS 70, and the PACS 90. Moreover, for example, the storage circuitry 13 stores computer programs causing support processing described below to be achieved. The storage circuitry 13 is implemented by, for example, a semiconductor memory element such as a random access memory (RAM) and a flash memory, a hard disk, an optical disk, or the like. Note that a storage area of the storage circuitry 13 may be in the support device 10 or in an external storage device connected by a network or other means. The storage circuitry 13 is an example of a storage unit.

The communication interface 15 controls transmission and communication of various data transmitted/received between the medical image diagnostic device 30, the HIS 50, the RIS 70, and the PACS 90. For example, the communication interface 15 receives image data from the medical image diagnostic device 30, the HIS 50, the RIS 70, or the PACS 90 and outputs the received data to the processing circuitry 11. For example, the communication interface 15 is implemented by a network card, a network adapter, a network interface controller (NIC), or the like.

The input interface 17 accepts various input operations from the operator, converts the accepted input operations into an electrical signal, and outputs the electrical signal to the processing circuitry 11. The input interface 17 accepts, for example, various input operations from the operator for various operation screens related to the support processing. As an example, the input interface 17 accepts an input operation related to the determination of a resection target site by the operator. Here, the input interface 17 is an example of an input unit.

For example, a mouse, a keyboard, a trackball, switches, buttons, a joystick, a touchpad, and a touch panel display can be used as the input interface 17, as appropriate. In the present embodiment, the input interface 17 is not limited to that including those physical operation components. For example, the input interface 17 also includes electrical signal processing circuitry that receives an electrical signal corresponding to an input operation from an external input device provided separately from the device and outputs the electrical signal to the processing circuitry 11. Moreover, the input interface 17 may be configured by a tablet terminal or the like capable of performing wireless communication with a main unit of the support device 10.

The display 19 displays various types of information. The display 19 outputs, for example, a graphical user interface (GUI) generated by the processing circuitry 11 and configured to accept various operations from the operator. The GUI configured to accept various operations from the operator includes various operation screens related to the support processing. For example, the display 19 outputs a display screen related to the support processing generated by the processing circuitry 11. As the display 19, various types of any desired display can be used as appropriate. For example, as the display 19, a liquid crystal display (LCD), a cathode ray tube (CRT), an organic electro luminescence (EL) display (OELD), or a plasma display can be used. The display 19 is an example of a display unit.

The display 19 may be of a desktop type, or may be configured by a tablet terminal or the like cable of performing wireless communication with the main unit of the support device 10. One or two or more projectors may be used as the display 19.

The processing circuitry 11 controls the entire operation of the support device 10. The processing circuitry 11 has a processor and a memory, such as ROM or RAM, as hardware resources. The processing circuitry 11 causes the processor configured to execute a computer program developed in the memory to execute an acquisition function 111, an analysis function 113, a calculation function 115, a learning function 117, and a display control function 119, and the like.

The processing circuitry 11 is an example of a processing unit. Processing circuitry 11 configured to implement the acquisition function 111 is an example of an acquisition unit. Processing circuitry 11 configured to implement the analysis function 113 is an example of an analysis unit. Processing circuitry 11 configured to implement the calculation function 115 is an example of a calculation unit. Processing circuitry 11 configured to implement the learning function 117 is an example of a learning unit. Processing circuitry 11 configured to implement the display control function 119 is an example of a display control unit.

In the acquisition function 111, the processing circuitry 11 acquires medical image data from the medical image diagnostic device 30, the HIS 50, the RIS 70 or the PACS 90 via the network 9. As an example, the processing circuitry 11 acquires medical image data depicting a target site of resection by surgical treatment. The target site of resection by surgical treatment is a target site of a procedure in which a lesion tissue or a target tissue for prophylactic resection is removed. As an example, in a case where the target site of resection by surgical treatment is at least part of a breast, the processing circuitry 11 acquires medical image data in a standing position or a sitting position. The processing circuitry 11 also acquires an input result of an operator accepted by the input interface 17. Note that the medical image data acquired from the medical image diagnostic device 30, the HIS 50, the RIS 70 or the PACS 90 may be two-dimensional image data or three-dimensional image data. Similarly, the relevant medical image diagnostic device 30 may be a device that generates two-dimensional image data, or may be a device that generates three-dimensional image data.

In the analysis function 113, the processing circuitry 11 analyzes a tissue composition of a target site based on the medical image data depicting the target site of resection by surgical treatment. As an example, in a case where the target site of resection by surgical treatment is at least part of a breast, the processing circuitry 11 analyzes a tissue composition, such as an amount of fat, mammary gland volume, blood vessel run and the like, for at least the part of the breast.

Note that in the present embodiment, treatment support with respect to breast reconstruction using adipose stem cells, that is, regenerative medicine about the breast is presented as an example, but the treatment support is not limited thereto. The treatment support according to the embodiments can be applied as appropriate to regenerative medicine for other anatomical structures (sites, organs) besides the breast. Other anatomical structures besides the breast may be a skin, a cardiac muscle, a blood vessel, a bone, or the like. As an example, a treatment support according to the present embodiment may be applied to skin reconstruction for skin diseases such as skin cancer. For example, with at least part of a skin as a target site, in the same manner as for the treatment support according to the embodiments, a resection site for at least the part of the skin can be estimated from images in advance, and an area and a volume of a cultivated skin related to the skin reconstruction can be calculated. As an example, the treatment support according to the present embodiment may be applied to bone reconstruction. For example, when a procedure to promote regeneration and engraftment with porous scaffold material and bone regenerative cells is performed on a patient with bone loss or aging who has difficulty in regenerating bone, the cell mass to be additionally produced for an additional surgical procedure can be calculated using X-ray images, CT images, MR images, and the like, in the same manner as for the treatment support according to the embodiments. As an example, the treatment support according to the present embodiment may be applied to cosmetic surgery or post-surgical plastic surgery. Other somatic stem cells besides adipose stem cells may also be used, depending on the anatomy and the treatment plan of the resection target. Pluripotent stem cells such as iPS cells may also be used as stem cells, not limited to somatic stem cells.

In the analysis function 113, the processing circuitry 11 also determines a resection target portion to be resected by surgical treatment, or a resection portion that was removed in surgical treatment. In other words, the analysis function 113 functions as a resection simulator that determines a resection target portion to be resected in surgical treatment or the resection portion that was resected in surgical treatment. As an example, the processing circuitry 11 determines a resection target portion or a resection portion in the medical image based on the output of the input interface 17 in response to the operation input of the operator. As the medical images, various medical images, such as X-ray CT images and MR images, can be used as appropriate.

As an example, in a case where a site of resection target in the surgical treatment is at least part of the breast, the operator, such as a doctor, for example, can use rendering image data (three-dimensional image data, volume data) in any desired viewpoint direction obtained by performing three-dimensional image processing such as volume rendering, surface rendering, image value projection processing, Multi-Planar Reconstruction (MPR) processing, and Curved MPR (CPR) processing, to set an area to be resected, such as an upper part inside the breast or an upper part outside the breast. In the analysis function 113, the processing circuitry 11 determines the resection area set by the operator as a resection target portion.

For example, in the analysis function 113, the processing circuitry 11 determines a resection portion resected in a surgical treatment based on the medical images before and after the resection in the surgical treatment. The medical images before and after resection are, for example, medical images of the same type, but may be medical images of different types. The type of medical image includes the type of the medical image diagnostic device 30 acquiring medical images, imaging conditions, patient postures during imaging, image processing, or the like. For example, a form can be implemented in which while X-ray CT images are used as medical images before resection, MR images or ultrasound images are used as medical images after the resection. Moreover, for example, a form can be implemented in which a medical image in a standing position is used as a medical image before resection, while a medical image in a sitting position is used as a medical image after the resection.

In the analysis function 113, the processing circuitry 11 may determine the resection target portion in a size that a predetermined margin is added to the resection area set by the operator. The predetermined margin may be predetermined and stored in the storage circuitry 13 or may be set in response to operation input by the operator. The processing circuitry 11 temporarily stores the volume and the tissue composition for the determined resection target portion or the resection portion in the storage circuitry 13.

In the calculation function 115, the processing circuitry 11 calculates, based on the determined resection target portion or the determined resection portion, the amount of cells to be produced. Specifically, the processing circuitry 11 determines, based on the composition of the tissue contained in the resection target portion or the resection portion, the type of cells (cell strain) to be produced. The processing circuitry 11 also calculates, based on the volume of the resection target portion or the resection portion, the amount of cells to be produced (cell mass). In other words, the processing circuitry 11 calculates the cell mass by cell strain to be produced based on the composition of the tissue contained in the resection target portion or the resection portion.

In the calculation function 115, the processing circuitry 11 calculates, based on the cell mass by cell strain to be produced, the time required to culture the cells to be introduced into the target site for resection in regenerative medicine. For example, the processing circuitry 11 estimates the amount of adipose stem cells that can be recovered from recovered from medical images. The processing circuitry 11 calculates, based on the cell mass by cell strain to be produced and the amount of adipose stem cells that can be recovered, the culture period required to obtain the cell mass to be produced. It is assumed that a relationship formula or a table indicating the correspondence between the cell mass and the culture time per stem cell is predetermined and stored in the storage circuitry 13 or the like, by cell strain.

In the learning function 117, the processing circuitry 11 learns a resection simulator that determines a resection target portion or a resection portion based on medical images before and after the resection in the surgical treatment. As an example, the processing circuitry 11 updates parameters of the resection simulator to output the resection target portion in response to input of the medical images and the resection target. As an example, the processing circuitry 11 updates the parameters of the resection simulator to output the resection portion in response to input of the medical images before and after the resection. As an example, the processing circuitry 11 updates a size of a predetermined margin that the resection simulator adds to a resection portion identified from a resection area set by the operator or identified from the medical images before and after the resection.

In the display control function 119, the processing circuitry 11 allows the display 19 to display a calculated cell type and cell mass for reconstruction.

Note that the support device 10 according to the embodiments can be mounted on a culture device for culturing cells. Alternatively, the support device 10 may be configured to output the calculated cell type and cell mass for reconstruction to the culture device.

Note that each of the functions 111, 113, 115, 117, and 119 is not necessarily implemented in a single processing circuitry. The processing circuitry 11 may be configured by combining a plurality of independent processors, and each of the functions 111, 113, 115, 117, and 119 may be implemented when the processor executes the corresponding computer program. The functions 111, 113, 115, 117, and 119 may be implemented by being integrated or distributed into a single processing circuitry or a plurality of pieces of processing circuitry as appropriate.

Note that the support device 10 is presented that allows a single computer to perform a plurality of functions as an example, but is not limited thereto. The plurality of functions of the support device 10 may be performed by separate computers. For example, the support device 10 may have a configuration in which at least two computers execute in a distributed manner the functions of the processing circuitry 11, such as the acquisition function 111, the analysis function 113, the calculation function 115, and the learning function 117.

The following describes support processing by the treatment support system 1 according to the embodiments with reference to the drawings.

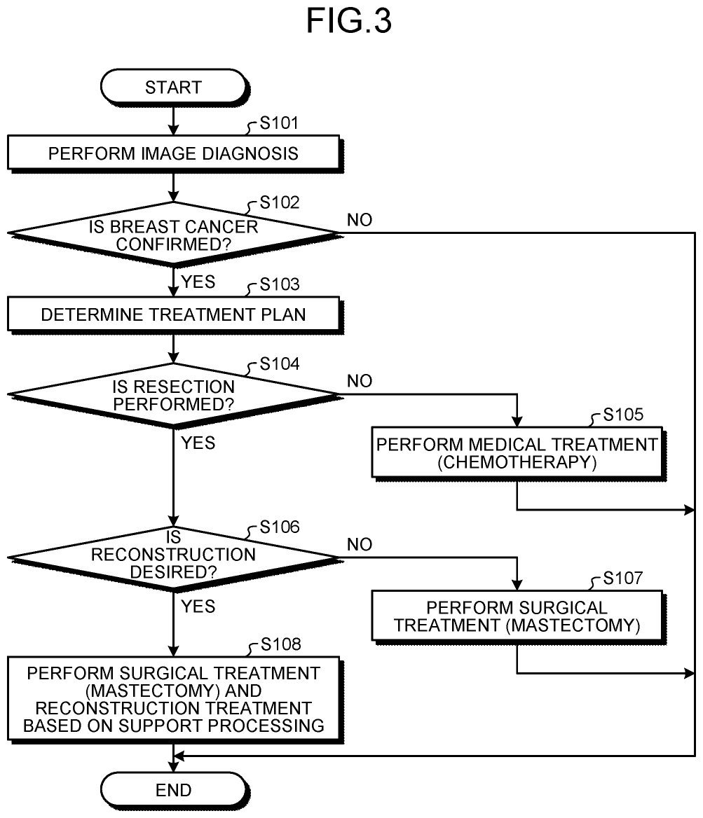

FIG. 3 is a flowchart illustrating an example of a workflow of the treatment support according to the embodiments.

First, image diagnosis based on medical images obtained by the medical image diagnostic device 30 is performed (S101). If breast cancer is confirmed as a result of image diagnosis (S102: Yes), a treatment plan is determined (S103). Here, if a treatment plan of not performing resection by surgical treatment is determined (S104: No), medical treatment (chemotherapy) is performed (S105). If a treatment plan of performing resection by surgical treatment (S104: Yes) and not desiring reconstruction (S106: No) is determined, surgical treatment (mastectomy) is performed (S107). If a treatment plan of performing resection by surgical treatment (S104: Yes) and desiring reconstruction (S106: Yes) is determined, surgical treatment (mastectomy) and reconstructive treatment based on support processing are performed (S108). If breast cancer is not confirmed as a result of image diagnosis (S102: No), or any treatment is performed, the workflow in FIG. 3 ends.

Note that in the present embodiment, a case where reconstructive treatment is performed when breast cancer is confirmed by diagnosis is presented as an example, but the case is not limited thereto. The workflow in FIG. 3 may be a flow of implementing reconstructive treatment when diagnosis is made to perform prophylactic resection.

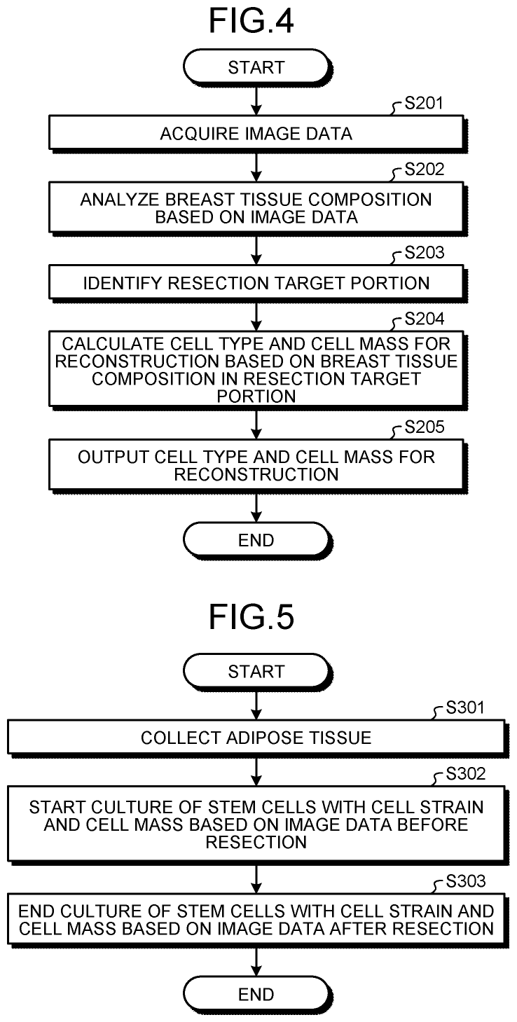

FIG. 4 is a flowchart illustrating an example of a flow of the support processing executed in the support device 10 according to the present embodiment.

The acquisition function 111 acquires medical image data from the medical image diagnostic device 30 and other devices (S201). The analysis function 113 analyzes a breast tissue composition based on the acquired medical image data (S202) and identifies a resection target portion based on an analysis result (S203). In other words, the analysis function 113 determines a resection target portion in a medical image depicting a target site of resection by surgical treatment. Subsequently, the calculation function 115 calculates cell type and cell mass for reconstruction based on the breast tissue composition of the resection target portion (S204). The display control function 119 outputs the calculated cell type and cell mass for reconstruction to display by the display 19 (S205).

Note that output of the cell type and cell mass for reconstruction includes cell type and cell mass calculated based on medical image data before resection and cell strain and cell mass calculated based on medical image data after resection. The cell strain and cell mass calculated based on medical image data after resection may be a difference with respect to the cell strain and cell mass calculated based on the medical image data before resection.

FIG. 5 is a flowchart illustrating an example of a workflow for cell culture in the treatment support according to the embodiments.

First, adipose tissue is collected from the subject (S301). Then, culture of stem cells is started with the cell strain and cell mass calculated based on the medical image data before resection (S302). Subsequently, when the cell culture is completed to the cell strain and the cell mass that have been calculated based on medical image data after resection, that is, that have been recalculated, the culture of stem cells is ended (S303).

Thus, the treatment support according to the embodiments includes determining a resection target portion in a medical image depicting a target site of resection by surgical treatment and calculating, based on the determined resection target portion, an amount of cells to be produced. As an example, the treatment support according to the embodiments includes analyzing a breast tissue composition from a medical image depicting a breast, and, in a case of an operation plan of performing reconstructive medicine, calculating cell type and cell mass required after resection, and culturing cells required for reconstruction.

With this configuration, an appropriate amount of cells can be supplied according to a reconstruction timing, and thus a breast reconstruction plan can be provided efficiently or systematically. In other words, with a treatment support according to the embodiments, the reconstruction of a target site by cell transplantation can be appropriately supported.

### Second Embodiment

In the treatment support according to the above-described embodiment, cell mass and cell type to be produced may be calculated further based on a take rate prospect of cultured cells introduced into the target site of resection by surgical treatment.

In the calculation function 115 according to the present embodiment, the processing circuitry 11 prospects the take rate of cultured cells introduced into the target site of resection by surgical treatment. Specifically, the processing circuitry 11 prospects an oxygen supply state of the cultured cells introduced, based on at least a size of a resection target portion or a resection portion and running of nutrient vessels therearound. Furthermore, the processing circuitry 11 prospects the take rate of cultured cells introduced by prospecting a volume of cells that could be necrotic after implantation, based on the prospected oxygen supply state. For example, the oxygen supply state decreases as a distance of the cultured cells from the nutrient vessels increases. In addition, the take rate becomes lower as the oxygen supply state decreases. Note that the processing circuitry 11 may prospect the take rate based on the distance between the cultured cells and the nutrient vessels without prospecting the oxygen supply state. It is assumed that a formula or a table indicating a relationship between the distance to the cultured cells from the nutrient vessels and the oxygen supply state or the take rate is predetermined and stored in the storage circuitry 13 or the like.

For example, in the calculation function 115, the processing circuitry 11 calculates cell mass to be produced in which the cell mass is corrected greatly compared to cell mass calculated based on a volume of a resection target portion or a resection portion as a prospected take rate is low. It is assumed that a formula or a table indicating a relationship between the take rate prospect and a correction amount of the cell mass are predetermined and stored in the storage circuitry 13.

For example, in the calculation function 115, the processing circuitry 11 adds vascular endothelial cells to the cell strains of cells to be produced to perform a vascular endothelial cell mixed transplantation when the prospected take rate is less than a predetermined threshold value. For example, cell mass of vascular endothelial cells to be produced is calculated based on a volume of cells that could be necrosed after being transplanted. Alternatively, the cell mass of vascular endothelial cells to be produced may be calculated based on the take rate. It is assumed that a threshold value of the take rate regarding whether to perform vascular endothelial cell mixed transplantation, and a formula or a table indicating a relationship between a volume or a take rate of cells that could be necrotic after being transplanted and the cell mass of vascular endothelial cells are predetermined and stored in the storage circuitry 13 or the like.

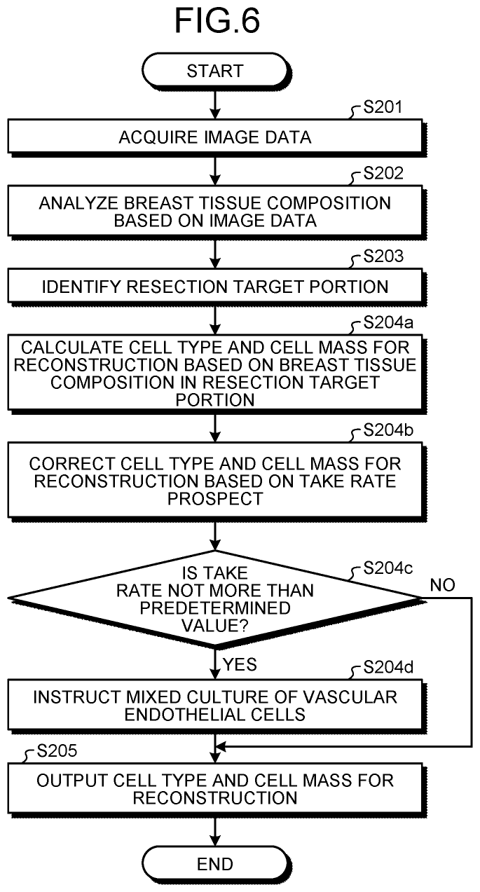

FIG. 6 is a flowchart illustrating another example of the flow of the support processing executed in the support device 10. A main difference from the flow in FIG. 4 is explained here.

The calculation function 115 first calculates cell type and cell mass for reconstruction (S204a) based on a breast tissue composition of a resection target portion similarly to the process at S204 in FIG. 4, and then prospects a take rate of cultured cells to be introduced into the target site of resection by surgical treatment, and based on the prospected take rate, corrects the cell type and the cell mass for reconstruction (S204b). Note that the calculation function 115 is not limited to correcting the cell type and the cell mass for reconstruction calculated at S204a, but may be configured to calculate the cell type and the cell mass for reconstruction based on the breast tissue composition and the take rate. If the take rate is not more than a predetermined threshold (predetermined value) (S204c: Yes), the display control function 119 instructs the operator to perform mixed culture of vascular endothelial cells (S204d). At this time, the calculation function 115 further calculates cell mass for vascular endothelial cells and outputs cell type and cell mass for reconstruction including the vascular endothelial cells, to display by the display 19 (S205). If the take rate is greater than the predetermined threshold (predetermined value) (S204c: No), the flow in FIG. 6 proceeds to the process at S205.

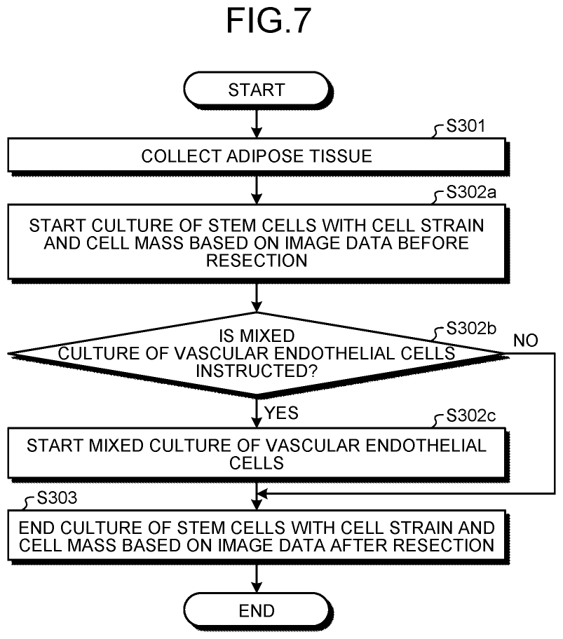

FIG. 7 is a flowchart illustrating another example of the workflow for cell culture in the treatment support according to the embodiments. A main difference from the flow in FIG. 5 is explained here.

When the culture of stem cells is started (S302a) with the cell strain and the cell mass calculated based on the medical image data before resection similarly to S302 in FIG. 5, if the mixed culture of vascular endothelial cells is instructed (S302b: Yes), the mixed culture of vascular endothelial cells is also started (S302c). Subsequently, when the cell culture is completed to the cell strain and the cell mass that have been calculated based on medical image data after resection, that is, that have been recalculated, the culture of stem cells is ended (S303).

Thus, the treatment support according to the present embodiment determines, further based on the take rate prospect of the cultured cells to be introduced into the target site, the cell type and the cell mass to be produced. With this configuration, an appropriate amount of cells can be supplied, taking into account necrosis of cultured cells after transplantation. In addition, it is possible to prevent the take rate of cells transplanted by the mixed culture of vascular endothelial cells from lowering.

### Third Embodiment

In the treatment support according to the above-described embodiment, the cultured cells introduced into the target site can be cultured using pre-stocked stem cells.

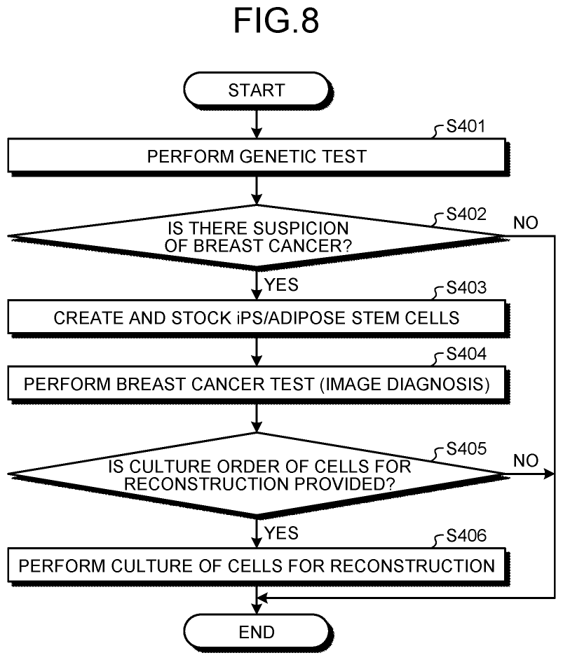

FIG. 8 is a flowchart illustrating another example of the workflow for cell culture in the treatment support according to the embodiment.

First, a genetic test of the subject is performed (S401). Samples for the genetic test may be circulating tumor cells or circulating tumor DNA derived from patients used in so-called liquid biopsy. If no breast cancer suspicion is diagnosed in the genetic test (S402: No), the flow in FIG. 8 ends. On the other hand, if breast cancer suspicion is diagnosed in the genetic test (S402: Yes), creation and stocking of stem cells are performed (S403). The stem cells may be somatic stem cells, such as adipose stem cells, or pluripotent stem cells, such as iPS cells. In parallel with or around the creation and stocking of stem cells, a breast cancer test (imaging test) is performed according to the workflow in FIG. 3 (S404). If a culture order of cells for reconstruction is provided based on the support processing in FIG. 4 or FIG. 6 (S405: Yes), the culturing cells for reconstruction is performed according to the workflow in FIG. 5 or FIG. 7 (S406).

In the calculation function 115 according to the present embodiment, the processing circuitry 11 calculates, based on the cell mass by cell strain to be produced and an amount of stock of at least one of iPS cells or adipose stem cells, a time required to culture cells to be introduced into the target site for resection in regenerative medicine.

Thus, in the treatment support according to the above-described embodiment, it is possible to shorten a time required to obtain an appropriate amount of cells by culturing cells to be introduced into the target site using stem cells that have been stocked in advance at any given timing, such as when a suspected breast cancer is diagnosed in a genetic test, for example.

### Fourth Embodiment

In the treatment support according to the above-described embodiment, in response to morphological changes over time after reconstruction (primary reconstructive procedure) of a target site such as a breast, additional cell transplantation (secondary reconstructive procedure) may be performed. Note that the secondary reconstruction may be performed more than once.

In the analysis function 113 according to the present embodiment, the processing circuitry 11 prospects volume reduction of cultured cells introduced in the primary reconstruction after the primary reconstruction. In other words, the analysis function 113 according to the present embodiment functions as a transitional simulator that prospects the volume reduction of the cultured cells introduced into the target site of resection by surgical treatment, and prospects changes in morphology over time after the reconstruction of the target site. The morphological changes over time are prospected based on, for example, cell mass and cell types that have been introduced, and an amount of absorption (amount of reduction) by cell type. It is assumed that a relational equation or a table indicating the amount of absorption (amount of reduction) by cell type per hour is predetermined and stored, for example, in the storage circuitry 13.

In the calculation function 115 according to the present embodiment, the processing circuitry 11 calculates the cell mass by cell strain required at the time of additional reconstruction (secondary reconstruction) based on a form of the target site after changes over time. More specifically, the processing circuitry 11 calculates the cell mass by cell strain required at the time of additional reconstruction (secondary reconstruction) based on the prospected result of the volume reduction of cultured cells by cell strain after the reconstruction.

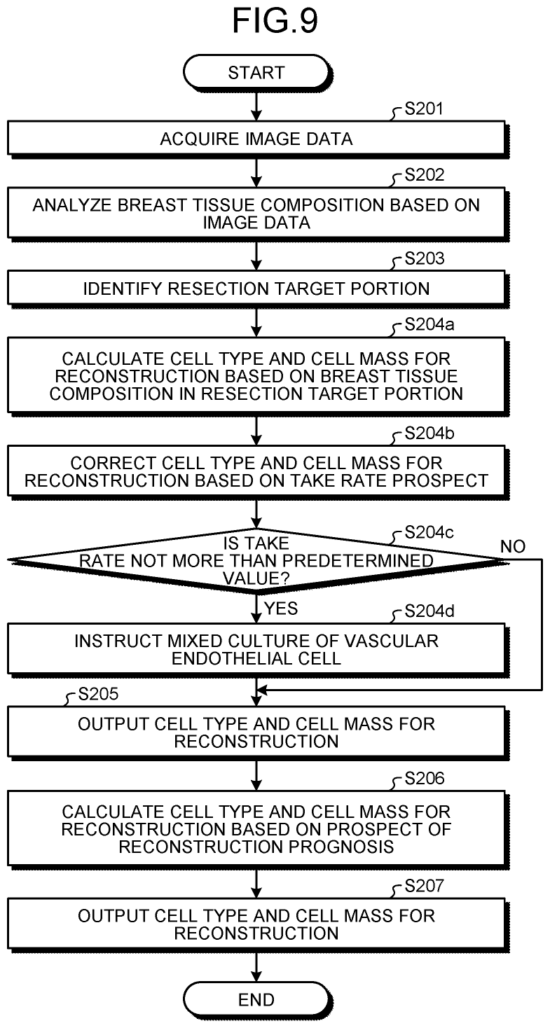

FIG. 9 is a flowchart illustrating another example of the flow of the support processing executed in the support device 10. While this section mainly describes differences from the flow in FIG. 6, steps can also be combined with the flow in FIG. 4.

The calculation function 115 calculates cell type and cell mass for additional reconstruction (S206) based on prospected results of morphological changes in reconstruction prognosis by the analysis function 113. The calculation function 115 then outputs the calculated cell type and cell mass for additional reconstruction to display by the display 19 (S207).

Note that processes at S206 and S207 do not need to be performed at different times from processes at S201 to S205, that is, do not need in succession with the processes at S201 to S205.

Note that the analysis function 113 may identify a form of the target site at the present time based on a medical image newly obtained after the primary reconstructive treatment. In this case, the calculation function 115 may calculate the cell mass by cell strain required at the time of additional reconstruction (secondary reconstruction) based on the form of the target site at the present time identified by the analysis function 113.

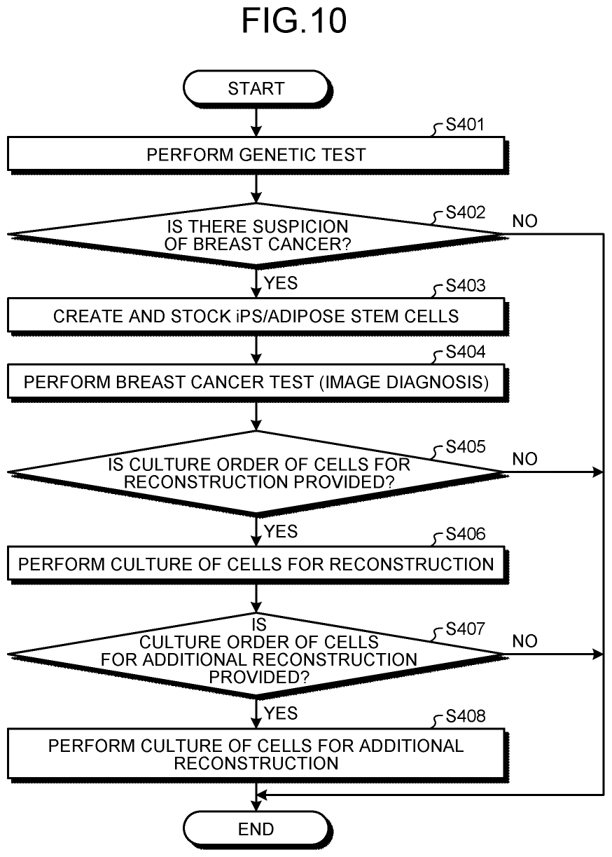

FIG. 10 is a flowchart illustrating another example of the workflow for cell culture in the treatment support according to the embodiment. A main difference from the flow in FIG. 8 is explained here.

After culture of cells for reconstruction is performed according to the workflow in FIG. 5 or FIG. 7 (S406), if a culture order of cells for additional reconstruction is provided based on the support processing in FIG. 9 (S407: Yes), the culture of cells for additional reconstruction is performed according to the workflow in FIG. 5 or FIG. 7 (S408). Note that the culture of cells for additional reconstruction at S408 can be started before the culturing for additional reconstruction at S406 ends.

Thus, in the treatment support according to the above-described embodiments, by calculating cell mass and cell type required for additional reconstruction based on prospect of morphological changes in the target site of the reconstruction prognosis, an appropriate amount of cells can be supplied according to an additional reconstruction timing, and thus breast reconstruction plan can be performed efficiently and in a planned manner. In other words, with the treatment support according to the embodiments, reconstruction processing that are performed more than once, including additional reconstruction of a target site by cell transplantation, can be appropriately supported.

### Fifth Embodiment

Note that while, in each of the above embodiments, reconstruction processing of a target site by regenerative medicine in which cultured cells are transplanted is presented as an example, reconstruction processing may be performed in combination with other reconstructive techniques of regenerative medicine.

Other reconstructive techniques combined with regenerative medicine may be autologous tissue reconstruction, such as a skin flap method or adipose injections, or artificial reconstruction, in which the skin is stretched with expanders and then replaced with implants. The skin flap method may be of a pedicle flap, in which tissue and blood vessels are moved without being resected, or a free flap, in which tissue is detached once and blood vessels are connected and implanted. The pedicle flap may be pedicle flap of latissimus muscle of back, in which a back tissue is transplanted, expansion flap of latissimus muscle of back, in which not only latissimus muscle of the back but also an adipose tissue of the waist are taken together, or rectus abdominis flap, in which a tissue from the abdomen is transplanted. A method of injecting adipose into the latissimus muscle of back to increase volume is also possible because adipose injected into the muscle is favorably grafted, and soft and large breasts can be reconstructed. The free flap may be free rectus abdominis flap or superficial inferior epigastric artery (SIEA flap).

The calculation function 115 reduces the amount of cells to be produced by tissue migration quantity by the skin flap method. Alternatively, the calculation function 115 reduces the amount of cells to be created by the amount of fat injected by fat injection. Alternatively, the calculation function 115 reduces the amount of cells to be produced by the volume of the implant to be implanted.

With this configuration, it is possible to expand the scope of regenerative treatment to which the treatment support according to each of the embodiments described above can be applied.

The term “processor” used in the above description means circuitry such as a central processing unit (CPU), a graphics processing unit (GPU), an application specific integrated circuit (ASIC), or a programmable logic device (PLD), for example. The PLD includes a simple programmable logic device (SPLD), a complex programmable logic device (CPLD), and a field programmable gate array (FPGA). The processor implements the functions by reading and executing the computer programs stored in the storage circuitry. The storage circuitry in which the computer programs are stored is a computer-readable, non-transitory recording medium. Note that instead of storing the computer programs in the storage circuitry, the computer programs may be directly incorporated in the circuitry of the processor. In this case, the processor implements the functions by reading and executing the computer programs incorporated in the circuitry. Instead of executing the computer programs, functions corresponding to the computer programs may be implemented by a combination of logic circuits. Each of the processors in the present embodiment is not limited to being configured as single piece of circuitry by processor, and one processor may be configured by combining a plurality of pieces of independent circuitry to implement the functions thereof. The plurality of components in FIG. 2 may be integrated into one processor to implement the functions thereof.

According to at least one of the embodiments described above, reconstruction of the target site by cell transplantation can be appropriately supported.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

With respect to the above embodiments, the following Appendices are disclosed as an aspect and selective feature of the invention.

(Appendix 1)

A medical information processing device including processing circuitry configured to:
determine a resection target portion in a medical image depicting a target site of resection by surgical treatment; and
calculate, based on the determined resection target portion, an amount of cells to be produced.

(Appendix 2)

The processing circuitry may calculate the amount of cells to be produced by cell strain based on a composition of a tissue contained in the resection target portion.

(Appendix 3)

The processing circuitry may prospect a take rate of cultured cells to be introduced into the target site and calculate the amount of cells to be produced further based on the prospected take rate.

(Appendix 4)

The processing circuitry may prospect the take rate based on a size and blood vessel run of the resection target portion.

(Appendix 5)

The processing circuitry may increase the amount of cells to be produced as the prospected take rate is low.

(Appendix 6)

The processing circuitry may add vascular endothelial cells to the cell strain to be produced when the prospected take rate is less than a predetermined threshold.

(Appendix 7)

The processing circuitry may calculate a time required to culture the cells to be produced based on a stock amount of at least one of iPS cells and adipose stem cells.

(Appendix 8)

The processing circuitry may determine a resection portion based on the medical image obtained after the resection by surgical treatment.

The processing circuitry may recalculate the amount of cells to be produced based on the determined resection portion.

(Appendix 9)

The processing circuitry may prospect morphological changes over time of a target site after reconstruction in which cultured cells are introduced into the target site of resection by surgical treatment.

The processing circuitry may calculate an amount of cells to be additionally produced based on a form of the target site after the prospected morphological changes.

(Appendix 10)

When the introduction of the cultured cells into the target site of resection by surgical treatment is combined with a skin flap method, the processing circuitry may reduce the amount of cells to be produced by tissue migration quantity by the skin flap method.

(Appendix 11)

The target site of resection by surgical treatment may be at least part of the breast. The medical image may be an image obtained in a standing posture or a sitting posture.

(Appendix 12)

The processing circuitry may determine, when the resection by surgical treatment is a partial resection, the resection target portion in a size obtained by adding a predetermined margin to a size of a resection target by the surgical treatment.

The processing circuitry may calculate the amount of cells to be produced for the resection target portion having the size including the added margin.

(Appendix 13)

A medical information processing method including:
determining a resection target portion in a medical image depicting a target site of resection by surgical treatment, and
calculating, based on the determined resection target portion, an amount of cells to be produced.

(Appendix 14)

A computer program for causing a computer to execute:
determining a resection target portion in a medical image depicting a target site of resection by surgical treatment, and
calculating, based on the determined resection target portion, an amount of cells to be produced.

(Appendix 15)

A computer program product having a computer program recorded therein, the computer program being executed by a computer, and being described in Appendix 14 described above.

What is claimed is:

1. A medical information processing device comprising processing circuitry configured to:
determine a resection target portion in a medical image depicting a target site of resection by surgical treatment, and
calculate, based on the determined resection target portion, an amount of cells to be produced.

2. The medical information processing device according to claim 1, wherein the processing circuitry calculates the amount of cells to be produced by cell strain based on a composition of a tissue contained in the resection target portion.

3. The medical information processing device according to claim 1, wherein the processing circuitry prospects a take rate of cultured cells to be introduced into the target site, and calculates the amount of cells to be produced further based on the prospected take rate.

4. The medical information processing device according to claim 3, wherein the processing circuitry prospects the take rate based on a size and blood vessel run of the resection target portion.

5. The medical information processing device according to claim 4, wherein the processing circuitry increases the amount of cells to be produced as the prospected take rate is low.

6. The medical information processing device according to claim 4, wherein the processing circuitry adds vascular endothelial cells to the cell strain to be produced when the prospected take rate is less than a predetermined threshold.

7. The medical information processing device according to claim 1, wherein the processing circuitry calculates a time required to culture the cells to be produced based on a stock amount of at least one of iPS cells and adipose stem cells.

8. The medical information processing device according to claim 1, wherein the processing circuitry
determines a resection portion based on the medical image obtained after the resection by surgical treatment, and
recalculates the amount of cells to be produced based on the determined resection portion.

9. The medical information processing device according to claim 1, wherein the processing circuitry
prospects morphological changes over time of a target site after reconstruction in which cultured cells are introduced into the target site of resection by surgical treatment, and
calculates an amount of cells to be additionally produced based on a form of the target site after the prospected morphological changes.

10. The medical information processing device according to claim 1, wherein when the introduction of the cultured cells into the target site of resection by surgical treatment is combined with a skin flap method, the processing circuitry reduces the amount of cells to be produced by tissue migration quantity by the skin flap method.

11. The medical information processing device according to claim 1, wherein
the target site of resection by surgical treatment is at least part of the breast, and
the medical image is an image obtained in a standing posture or a sitting posture.

12. The medical information processing device according to claim 1, wherein the processing circuitry
determines, when the resection by surgical treatment is a partial resection, the resection target portion in a size obtained by adding a predetermined margin to a size of a resection target by the surgical treatment, and
calculates the amount of cells to be produced for the resection target portion having the size including the added margin.

13. A medical information processing method comprising:
determining a resection target portion in a medical image depicting a target site of resection by surgical treatment, and
calculating, based on the determined resection target portion, an amount of cells to be produced.

14. A computer program product comprising instructions that cause a computer to execute:
determining a resection target portion in a medical image depicting a target site of resection by surgical treatment; and
calculating, based on the determined resection target portion, an amount of cells to be produced based on the determined resection target portion.
